(19) United States
(12) Patent Application Publication
(10) Pub. No.: US 2004/0165872 A1
(43) Pub. Date: Aug. 26, 2004

(54) FUNDUS CAMERA

(76) Inventors: Tsuguo Nanjo, Toyohashi-shi (JP); Kunihiko Konoma, Hoi-gun (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/782,817

(22) Filed: Feb. 23, 2004

(30) Foreign Application Priority Data
Feb. 26, 2003 (JP) JP2003-49598

(51) Int. Cl. A61B 3/14

(52) U.S. Cl. 396/18

(57) ABSTRACT

A fundus camera capable of guiding accurately a sight line using an internal fixation target light source while an apparatus constitution is simplified. The fundus camera is provided with an illumination optical system for illuminating the fundus with visible illumination light for photographing and with invisible illumination light for observation, a photographing optical system for picking up an image of the fundus with visible reflection light from the fundus including a first image-pickup element, an observation optical system for picking up the image of the fundus with invisible reflection light from the fundus including a wavelength-selecting mirror and a second image-pickup element, a fixation target projection optical system for fixating an eye including alight-dividing member and a fixation target light source, and a wavelength-selecting filter. In the fundus camera, the visible fixation target light is guided to the eye and photo-received on the second image-pickup element.

### BACKGROUND OF THE INVENTION

1. Field of the Invention

The present invention relates to a fundus camera for photographing a fundus of an eye to be examined.

2. Description of Related Art

There is known a fundus camera which photographs a fundus under a non-mydriasis condition without using mydriatics. Conventionally, for the fundus camera in this type of fundus camera, a pop-up mirror having a regular mirror coating is used to switch between an optical path of an observation optical system which observes the fundus in invisible infrared light and an optical path of a photographing optical system which photographs the fundus under visible light emitted from a flash light or the like. However, a mechanism using the pop-up mirror has disadvantages of inevitable driving sounds, vibrations and the like at the time of photographing in addition to necessity of a control sequence for synchronizing and driving the pop-up mirror with the flash light or the like, and complication of a driving mechanism. Therefore, it is proposed that a dichroic mirror having a wavelength-selecting property is provided instead of the pop-up mirror to guide the visible light to the optical path of the photographing optical system and the infrared light to the optical path of the observation optical system respectively.

In addition, in the fundus camera of non-mydriasis type, it is preferable that a fixation target light source for guiding a sight line of an examinee (a patient) is arranged in an optical system thereof. It is also preferable that an image of the fixation target light source is observable by an examiner. Consequently, it is proposed that a light-dividing member is provided on the optical path of the observation optical system and a reflection mirror is further provided to reflect light emitted from the fixation target light source and reflected by the light-dividing member toward an image-pickup element in the observation optical system, and the image of the fixation target light source is superimposed on an image of the fundus to be displayed on a monitor.

However, an apparatus is disadvantageously upsized when the reflection mirror for reflecting the light emitted from the fixation target light source is provided in the optical system. Especially for a fundus camera of handheld type, the upsizing of the apparatus is a big problem.

### SUMMARY OF THE INVENTION

The present invention has been made in view of the above circumstances and an object to overcome the above problems and to provide a fundus camera capable of guiding accurately a sight line using an internal fixation target light source while an apparatus constitution is simplified.

To achieve the objects and in accordance with the purpose of the present invention, a fundus camera for photographing a fundus of eye to be examined, the camera is provided with an illumination optical system for illuminating the fundus with visible illumination light for photographing and with invisible illumination light for observation, a photographing optical system including a first image-pickup element which has sensitivity to a visible range for picking up an image of the fundus with visible reflection light from the fundus, an observation optical system for picking up the image of the fundus with invisible reflection light from the fundus including a wavelength-selecting mirror having a wavelength-selecting property of reflecting approximately all light within an invisible range and a part of light within the visible range and transmitting a large residual part of the light within the visible range disposed on a first optical path of the photographing optical system and a second image-pickup element having sensitivity to the visible range and invisible range disposed on a second optical path divided from the first optical path by the wavelength-selecting mirror, a fixation target projection optical system for fixating an eye including a light-dividing member disposed on the second optical path of the observation optical system and a fixation target light source for emitting visible fixation target light disposed in an approximately conjugate position with an image-pickup surface of the second image-pickup element on a third optical path divided from the second optical path by the light-dividing member, and a wavelength-selecting filter having a wavelength-selecting property of transmitting approximately all of the invisible range and a part of the visible range and reflecting another part of the visible range disposed on the second optical path between the wavelength-selecting mirror and the light-dividing member in an approximately conjugate position with the image-pickup surface of the second image-pickup element. In the fundus camera, the visible fixation target light is guided to the eye and photo-received on the second image-pickup element.

Additional objects and advantages of the invention are set forth in part in the description which follows and in part will be obvious from the description, or may be learned by practice of the invention. The objects and advantages of the invention may be realized and attained by the fundus camera in the claims.

### BRIEF DESCRIPTION OF THE DRAWINGS

The accompanying drawings, which are incorporated in and constitute a part of this specification, illustrate an embodiment of the present invention and, together with the description, serve to explain the objects, advantages and principles of the invention. In the drawings,

FIG. 1 is a view showing a schematic configuration of a fundus camera;

FIG. 2 is a view showing a schematic configuration of an optical system of the fundus camera;

FIGS. 3A and 3B are views illustrating optical characteristics of a dichroic mirror and a wavelength-selecting filter;

FIG. 4 is a schematic block diagram of a control system in the fundus camera; and

FIG. 5 is a view showing an example of a display screen on a monitor in an alignment mode.

### DETAILED DESCRIPTION OF THE PREFERRED EMBODIMENTS

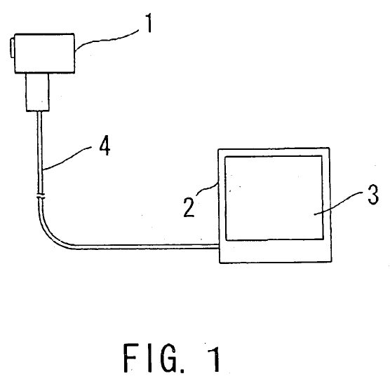

A detailed description of one preferred embodiment of a fundus camera consistent with the present invention will now given referring to the accompanying drawings. FIG. 1 is a view showing a schematic configuration of a fundus camera of non-mydriasis type in consistent with the preferred embodiment of the present invention.

The fundus camera according to the embodiment is roughly constituted of a photographing unit 1, in which optical systems for observation and photographing are contained in a housing suitable for handheld operation, and a control unit 2 by which the photographing unit 1 is controlled and in which a light source for photographing is disposed. The control unit 2 is installed with a color LCD monitor 3 with a touch-panel function. Illumination light emitted from the light source for photographing of the control unit 2 is directed to the optical system in the photographing unit 1 via an optical fiber 4.

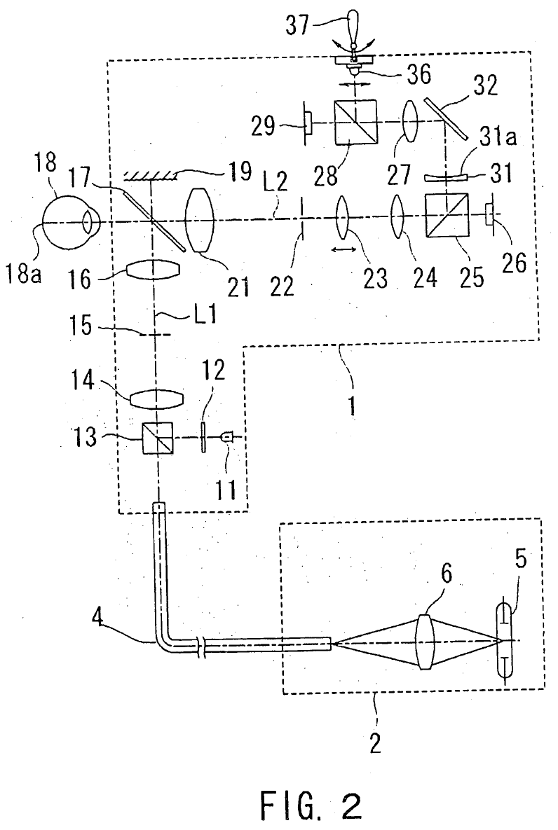

FIG. 2 is a view showing a schematic configuration of an optical system of the fundus camera. The optical system is constituted of an illumination optical system for observation, an illumination optical system for photographing, an observation/photographing optical system, and a fixation target projection optical system.

Illumination Optical System for Observation

Infrared illumination light with a central wavelength of approximately 880 nm, which is emitted from an infrared light source 11 for observation such as an infrared LED or the like, is reflected by a dichroic mirror 13 via a diffusing plate 12. The dichroic mirror 13 has a wavelength-selecting property of reflecting approximately all light within an infrared range and transmitting approximately all light within a visible range. The infrared illumination light reflected by the dichroic mirror 13 passes through a condenser lens 14, a slit plate 15 and a projection lens 16, and then a part of the light is reflected by a half mirror 17 to be projected onto an eye 18 to be examined. The slit plate 15 is provided with a pinhole opening (aperture) at its center (on an optical axis L1) and a ring-slit opening (aperture) around the pinhole opening. The half mirror 17 makes the optical axis L1 of the illumination optical system coaxial with an optical axis L2 in the observation/photographing optical system described later. A black absorber 19 absorbs illumination light transmitted through the half mirror 17 so as to prevent unnecessary noise light from entering the observation/photographing optical system. Incidentally, instead of the infrared light source 11, a halogen light and an infrared transmission filter, or the like may be used.

Illumination Optical System for Photographing

Visible illumination light emitted from a visible light source 5 for photographing such as a flash light goes via a condenser lens 6 and is directed through the optical fiber 4 to the photographing unit 1. The visible illumination light directed to the photographing unit 1 is transmitted through the dichroic mirror 13, and passes through the condenser lens 14 to the half mirror 17 to be projected onto the eye 18.

Observation/Photographing Optical System

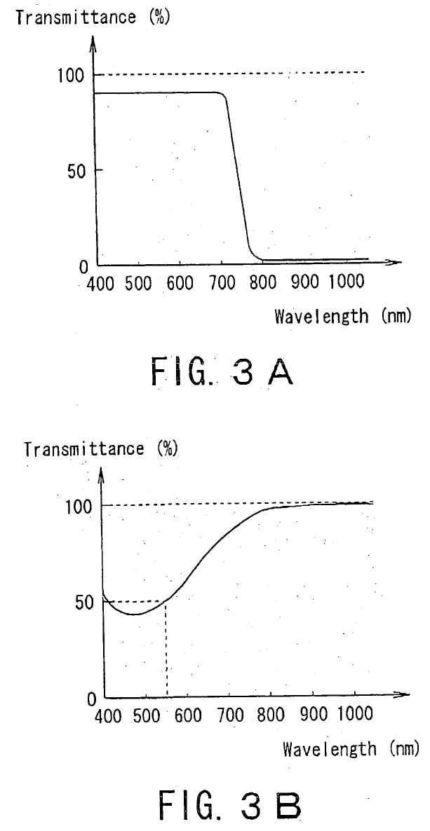

Infrared reflection light and visible reflection light from the eye 18 go via the half mirror 17, an objective lens 21, a photographing diaphragm 22, a focusing lens 23 and an image forming lens 24 to be entered a dichroic mirror 25. The photographing diaphragm 22 is disposed in an approximately conjugate position with a pupil of the eye 18. The focusing lens 23 is disposed to be movable along the optical axis L2 by an unillustrated lens moving mechanism for performing adjustment in accordance with refractive power of the eye 18. As shown in FIG. 3A, the dichroic mirror 25 has a wavelength-selecting property of reflecting approximately all light within the infrared range of approximately 800 nm or more, and reflecting a part (approximately 10% to approximately 20%) of light within the visible range of approximately 700 nm or less to approximately 800 nm or less and transmitting a large residual part (approximately 90% to approximately 80%) of the light within the visible range. The visible reflection light transmitted through the dichroic mirror 25 is photo-received on a CCD camera 26 for photographing which has sensitivity to the visible range to form an image of the eye 18 thereon. In addition, the infrared reflection light reflected by the dichroic mirror 25 goes via a wavelength-selecting filter 31, a total reflection mirror 32 and a relay lens 27, and is transmitted through a dichroic mirror 28, and then is photo-received on an CCD camera 29 for observation to form an image of the eye 18 thereon. The dichroic mirror 28 has a wavelength-selecting property of transmitting approximately all the light within the infrared range of approximately 800 nm or more, and transmitting a part (approximately 10% to approximately 20%) of the light within the visible range of approximately 700 nm or less to approximately 800 nm or less and reflecting a large residual part (approximately 90% to approximately 80%) of the light within the visible range. Besides, the filter 31 will be described later.

Incidentally, the ratio between reflection and transmission of the dichroic mirrors 25 and 28 are not particularly limited to that described above, and a variety of modifications may be made.

Fixation Target Projection optical System

Visible fixation target light within a narrow wavelength range having a central wavelength of approximately 550 nm emitted from a fixation target light source 36 such as a visible LED is reflected by the dichroic mirror 28 and enters the dichroic mirror 25 via the relay lens 27, the mirror 32, and the filter 31. A part of the visible fixation target light which has entered the dichroic mirror 25 is reflected thereby and projected onto the eye 18, after passing through image forming lens 24 to the half mirror 17, so that the eye 18 may visually identify the fixation target light source 36.

Incidentally, the fixation target light source 36 is disposed at an end of an adjustment knob 37 and its position is arranged to be changeable within an approximately conjugate plane with a fundus 18a of the eye 18 and an image-pickup surface of the CCD camera 29. The fixation target light source 36 is moved within a plane approximately vertical to an optical axis thereof as an examiner operates the adjustment knob 37. Accordingly, the position of the fixation target light source 36 presented to the eye 18 is changed, and the fundus 18a may be guided to a desired photographing position.

The filter 31 is disposed in a position, between the dichroic mirror 25 and the dichroic mirror 28, where an image of the fundus 18a is approximately formed (i.e. an approximately conjugate position with the image-pickup surface of the CCD camera 29). As shown in FIG. 3B, the filter 31 has a wavelength-selecting property of transmitting approximately all the light within the infrared range of approximately 800 nm or more, and transmitting approximately half of light within the visible range of around approximately 550 nm and reflecting the residual light.

Besides, various modifications of the ratio of reflection to transmission of the filter 31 are possible since the ratio is not limited to that specific ratio. In addition, the wavelength of the fixation target light is neither limited to that mentioned above, and the wavelength-selecting property of the filter 31 is determined in accordance with the wavelength of the fixation target light.

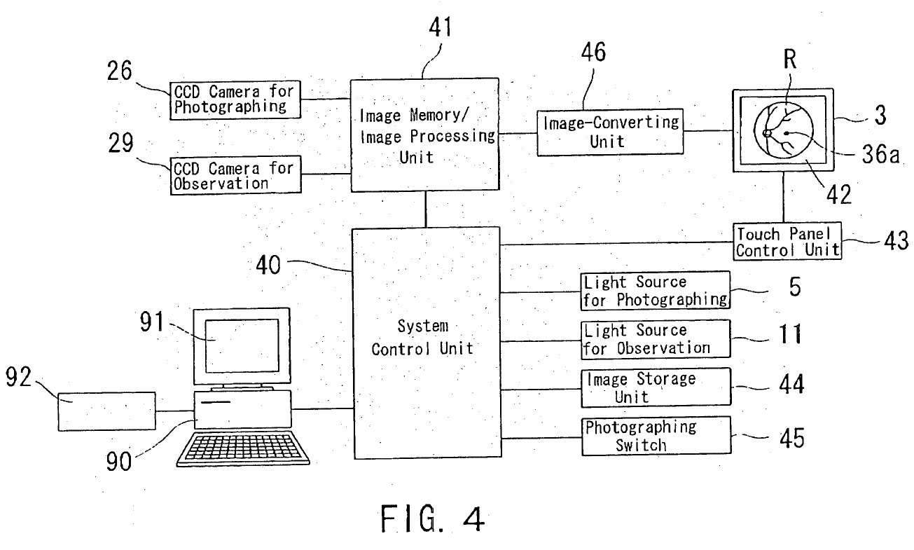

Since the filter 31 reflects approximately half of the visible fixation target light from the fixation target light source 36, an image of the fixation target light source 36 is formed on the image-pickup surface of the CCD camera 29, and the image R of the fundus 18a and the image 36a of the fixation target light source 36 are together displayed on the monitor 3 to which an output of the CCD camera 29 is connected (see FIG. 4). Thereby, the fixation target light source 36 may be moved while observed on the monitor 3.

Incidentally, in the filter 31, a reflection surface 31a on a mirror 32 side which reflects the visible fixation target light from the fixation target light source 36 is concavely formed (or a surface on a dichroic mirror 25 side may be concavely formed), the filter 31 may efficiently reflect the light from the fixation target light source 36 even in a case where the fixation target light source 36 has been moved.

FIG. 4 is a schematic block diagram of a control system in the present apparatus. Outputs from the CCD camera 29 and the CCD camera 26 are inputted into an image memory/image-processing unit 41. Images inputted into the image memory/image-processing unit 41 are displayed on the monitor 3 via an image-converting unit. The monitor 3 is provided with a touch panel 42, and detection of a touched position and the like are carried out through a touch-panel control unit 43. Connected with a system control unit 40 are the image memory/image-processing unit 41, the touch-panel control unit 43, an image storage unit 44, the light source 5 for photographing, the light source 11 for observation, a photographing switch 45, and an external computer 90 and the like.

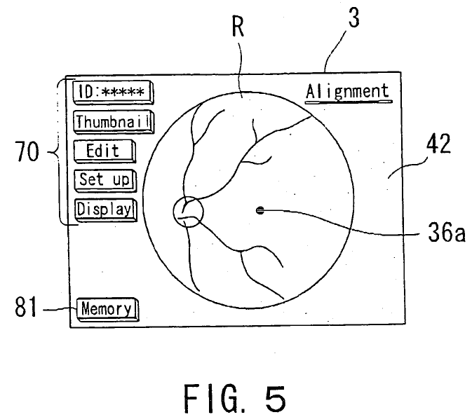

Next, in the fundus camera having the above-mentioned constitution, a description on operation thereof will be given. An initial screen when the power is turned on is placed in an alignment mode. FIG. 5 shows an example of a display screen given on the monitor 3 in the alignment mode. The monitor 3 is provided with the touch panel 42, and a group of touch keys 70 having a variety of functions and a touch key 81 to store images are displayed thereon. When an operator touches the touch key on the touch panel 42 with his/her finger and the like, the touched position is detected by the touch-panel control unit 43 and a predetermined operational signal is inputted.

The examiner performs alignment of the optical system of the photographing unit 1 with the eye 18 when the power is turned on, the system control unit 40 lights the light source 11 for observation. The infrared illumination light emitted from the light source 11 is uniformized by the diffusing plate 12, and then is reflected by the dichroic mirror 13 and converged at the condenser lens 14 to illuminate the whole surface of the slit plate 15. The infrared light passing through the opening of the slit plate 15 goes via the projection lens 16 and enters the half mirror 17 where intensity of the light is attenuated to about a half and reflected, and then the attenuated light is projected onto the eye 18. When the photographing unit 1 is positioned at a predetermined working distance, the infrared light restricted in a ring shape through the ring-slit opening of the slit plate 15 once forms an image of the ring slit in a vicinity of the pupil of the eye 18, and then diffuses to illuminate the fundus 18a in a field of view which is as large as or slightly larger than a field of view to be photographed.

The infrared reflection light from the fundus 18a passes through the half mirror 17, the objective lens 21, the photographing diaphragm 22, the focusing lens 23 and the image forming lens 24, and then it is reflected by the dichroic mirror 25. After that, it is converged by the relay lens 27 via the filter 31 and the mirror 32, transmitted through the dichroic mirror 28, and forms an image on the image-pickup element of the CCD camera 29.

After undergoing A/D conversion, a picture signal from the CCD camera 29 is inputted into the monitor 3 through the image memory/image-processing unit 41 and an image converting unit 46 for converting the picture signal into an image signal for an LCD, and the image of the eye 18 is displayed thereon. While observing the image of the eye 18 displayed on the monitor 3 (at this point, the image is in monochrome) and observing an alignment reflex formed by an unillustrated alignment optical system (which, for example, may have a constitution that exit end faces of a pair of bilateral optical fibers which emit infrared light are arranged in front of the photographing diaphragm 22), the examiner performs alignment for adjusting a working distance between the eye 18 and the optical systems and optical axis adjustment. After completing the alignment, the focusing lens 23 is moved so that the image-pickup surfaces of the CCD cameras 26 and 29 are in conjugate positions with the fundus 18a respectively.

When a photographing portion of the fundus 18a is to be moved, the adjustment knob 37 is operated to move the fixation target light source 36 to a desired photographing position. Approximately half of the visible fixation target light from the fixation target light source 36 is transmitted through the filter 31 and the residual half is reflected thereby. Since the fixation target light source 36 and the image-pickup surface of the CCD camera 29 are in a conjugate positional relation and the filter 31 is disposed in the approximately conjugate position with the image-pickup surface of the CCD camera 29, the image of the fixation target light source 36 is formed on the image-pickup element and the image 36a of the fixation target light source 36 is displayed together with the image R of the fundus on the monitor 3 to which the output of the CCD camera 29 is connected. The examiner may easily guide the sight line of the examinee while observing the fixation target image 36a on the monitor 3.

In a condition where focus adjustment is completed and image of the fundus to be photographed is observable, the examiner depresses the photographing switch 45 to generate a trigger signal. The control unit 40, in which the trigger signal is inputted, controls to light the light source 5 for photographing in order to illuminate the fundus 18a with the visible illumination light.

As well as the infrared reflection light, the visible reflection light from the fundus 18a enters the dichroic mirror 25, after passing through the half mirror 17, the objective lens 21, the photographing diaphragm 22, the focusing lens 23 and the image forming lens 24. As the dichroic mirror 25 transmits a large part of the visible light, the visible reflection light forms an image on the image-pickup element of the CCD camera 26 through the image forming lens 24. A picture signal from the CCD camera 26 is inputted into the image memory/image processing unit 41 and stored in the image memory/image processing unit 41 as a still image in synchronization with the lighting of the light source 5.

Additionally, under control of the control unit 40, the picture signal from the CCD camera 26 is inputted into the monitor 3 via the image converting part 46, and the image of the fundus 18a (at this point, the image is in color) is displayed thereon.

Connected with the control unit 40 is the image storage unit 44 including an MO (magneto-optic disk), a memory card or the like for storing a large amount of image data. The touch key 81 on the touch panel 42 is depressed, and a photographed image captured by the image memory/image processing unit 41 is stored in the image storage unit 44.

When panoramic image is to be obtained by photographing a plurality of images of the fundus, the adjustment knob 37 is operated to move the fixation target light source 36 to a desired position for photographing and the images of the fundus are photographed in the same way as mentioned above.

Data on the image captured by the image memory/image processing unit 41 and data on the image stored in the image storage unit 44 may be sent to and outputted from the external computer 90 connected with a communication cable, and may be displayed on a display 91 of the external computer 90 and printed out by a printer 92.

As explained above, according to the present invention, the sight line may be guided accurately through the internal fixation target light source while the apparatus constitution is simplified. Especially for a fundus camera of handheld type, an advantageous constitution may be obtained.

The foregoing description of the preferred embodiments of the invention has been presented for purposes of illustration and description. It is not intended to be exhaustive or to limit the invention to the precise form disclosed, and modifications and variations are possible in the light of the above teachings or may be acquired from practice of the invention. The embodiments chosen and described in order to explain the principles of the invention and its practical application to enable one skilled in the art to utilize the invention in various embodiments and with various modifications as are suited to the particular use contemplated. It is intended that the scope of the invention be defined by the claims appended hereto, and their equivalents.

What is claimed is:

1. A fundus camera for photographing a fundus of an eye to be examined, the fundus camera comprising:
an illumination optical system for illuminating the fundus with visible illumination light for photographing and with invisible illumination light for observation;
a photographing optical system, including a first image-pickup element which has sensitivity to a visible range, for picking up an image of the fundus with visible reflection light from the fundus;
an observation optical system for picking up the image of the fundus with invisible reflection light from the fundus, the observation optical system including
a wavelength-selecting mirror having a wavelength-selecting property of reflecting approximately all light within an invisible range and a part of light within the visible range and transmitting a large residual part of the light within the visible range, the wavelength-selecting mirror being disposed on a first optical path of the photographing optical system, and
a second image-pickup element having sensitivity to the visible range and the invisible range, the second image-pickup element being disposed on a second optical path divided from the first optical path by the wavelength-selecting mirror;
a fixation target projection optical system for fixating an eye, the fixation target projection optical system including
a light-dividing member disposed on the second optical path of the observation optical system, and
a fixation target light source for emitting visible fixation target light, the fixation target light source-being disposed in an approximately conjugate position with an image-pickup surface of the second image-pickup element on a third optical path divided from the second optical path by the light-dividing member; and
a wavelength-selecting filter having a wavelength-selection property of transmitting approximately all the light within the invisible range and a part of light within the visible range and reflecting another part of the light within the visible range, the wavelength-selecting filter being disposed on the second optical path between the wavelength-selecting mirror and the light-dividing member in an approximately conjugate position with the image-pickup surface of the second image-pickup element,
wherein the visible fixation target light is guided to the eye and photo-received on the second image-pickup element.

2. The fundus camera according to claim 1, wherein the wavelength-selecting mirror reflects approximately 10% to approximately 20% of the light within the visible range.

3. The fundus camera according to claim 1, wherein the wavelength-selecting filter transmits approximately half of the light within the visible range.

4. The fundus camera according to claim 1, wherein
the fixation target light source emits the fixation target light within a narrow visible range having a predetermined central wavelength, and
the wavelength-selecting filter transmits a part of the light within the narrow visible range having the predetermined central wavelength.

5. The fundus camera according to claim 1, wherein a reflection surface of the wavelength-selecting filter is formed to be a concave surface.

6. The fundus camera according to claim 1, wherein the illumination optical system includes an illumination optical system for photographing which projects the visible illumination light for photographing and an illumination optical system for observation which projects the infrared illumination light for observation.

7. The fundus camera according to claim 1, further comprising a moving unit which moves the fixation target light source within a plane approximately vertical to an optical axis of the fixation target projection optical system.
